(19) United States
(12) Patent Application Publication
(10) Pub. No.: US 2019/0388157 A1
(43) Pub. Date: Dec. 26, 2019

(54) SURGICAL NAVIGATION SYSTEM WITH PATTERN RECOGNITION FOR FAIL-SAFE TISSUE REMOVAL

(71) Applicant: Acclarent, Inc., Irvine, CA (US)

(72) Inventors: Ehsan Shameli, Irvine, CA (US); Fatemeh Akbarian, Rancho Palos Verdes, CA (US); Babak Ebrahimi, Irvine, CA (US)

(21) Appl. No.: 16/404,982

(22) Filed: May 7, 2019

Related U.S. Application Data
(60) Provisional application No. 62/687,861, filed on Jun. 21, 2018.

(51) Int. Cl. A61B 34/20; A61B 34/00; A61B 34/10

(52) U.S. Cl. CPC A61B 34/20; A61B 34/25; A61B 2017/00119; A61B 34/76; A61B 2034/2065; A61B 34/10

(57) ABSTRACT

A physician preparing to perform a surgical procedure may view pre-operative images of a procedure area of a patient and use a stylus or other interface to draw boundaries between or around areas shown in the images that should be treated with extreme caution during the procedure. These boundaries may be provided by an image guided surgery navigation system and used to provide warnings (e.g., audible or visual alerts having characteristics associated with the proximity to the bounded area) and control operations of a medical instrument (e.g., reducing a cutting speed or disabling the medical instrument entirely) during the procedure in order to reduce risks associated with using the medical instrument in or around the bounded areas. Other safety features that may be defined or pre-configured based on the procedure include reacting to movement patterns (e.g., high linear speed) and location patterns (e.g., steady advancement towards an unsafe area).

### PRIORITY

This application claims priority to U.S. Provisional Pat. App. No. 62/687,861, entitled “Surgical Navigation System with Pattern Recognition for Fail-Safe Tissue Removal,” filed Jun. 21, 2018, the disclosure of which is incorporated by reference herein.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation systems that may be used in IGS procedures is the CARTO® 3 System by Biosense-Webster, Inc., of Irvine, Calif.. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

Surgical cutting instruments configured for removal of lesions, polyps and fibroids within the nasal cavity or other procedure areas may include an elongated inner member rotatably coaxially disposed within a tubular outer member. The distal end of the outer member may include an opening, and the distal end of the inner member may include corresponding cutting edges. Position and orientation of the cutting edges are important for the success of a procedure, so an IGS navigation system may also be particularly useful in procedures involving surgical cutting instruments being used in procedure areas of limited visibility, such as within the nasal cavity of a patient. Even where IGS navigation systems are used with medical instruments such as surgical cutting instruments, there is still a possibility of error that could impact a procedure outcome, whether the source is human error (e.g., an unintentional movement of the medical instrument or an intentional but erroneous movement, etc.) or equipment error (e.g., obscurement or failure of an endoscopic view, failure of a display of an IGS navigation system, etc.). Thus, it may be advantageous to provide an IGS navigation system with additional safety features to further reduce the risks associated with surgical procedures.

While several systems and methods have been made and used in surgical procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary surgery navigation system being used on a patient seated in an exemplary medical procedure chair;

FIG. 2 depicts a perspective view of an exemplary surgical cutting instrument having a handle assembly and a first shaft assembly;

FIG. 3 depicts an exploded perspective fragmentary view of the shaft assembly of FIG. 2 having a shaft and a cutting member;

FIG. 4 depicts a schematic view of an exemplary system configured to provide one or more safety features during a surgical procedure using the surgery navigation system of FIG. 1 and the surgical cutting instrument of FIG. 2;

FIG. 5 depicts an exemplary set of high level steps that may be performed by the system of FIG. 4 to provide the one or more safety features;

FIG. 6 depicts an exemplary set of steps that may be performed by the system of FIG. 4 to configure the one or more safety features;

FIG. 7 depicts an exemplary set of steps that may be performed by the system of FIG. 4 to monitor for conditions that are covered by the one or more safety features;

FIG. 8 depicts an exemplary set of steps that may be performed by the system of FIG. 4 to address conditions that are covered by the one or more safety features;

FIG. 9 depicts a screenshot of an exemplary interface that a user may use when configuring the one or more safety features;

FIG. 10 depicts another screenshot of an exemplary interface that a user may use when configuring the one or more safety features; and

FIG. 11 depicts a graph showing a set of data generated from a medical instrument during a surgical procedure that could be used to identify unsafe conditions and enforce one or more safety features.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

I. Exemplary Image Guided Surgery Navigation System

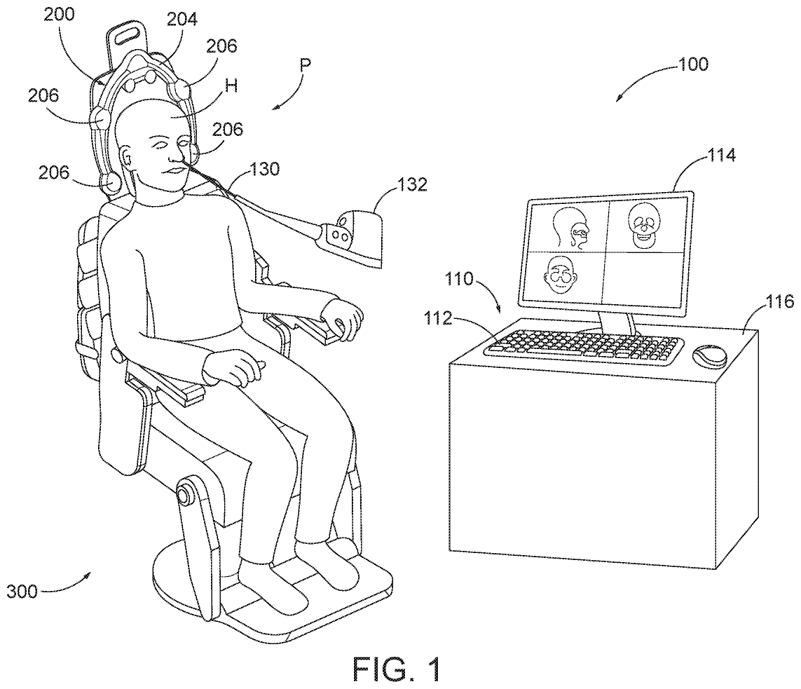

FIG. 1 shows an exemplary IGS navigation system (100) enabling an ENT procedure to be performed using image guidance. In some instances, IGS navigation system (100) is used during a surgical procedure involving the use of a surgical cutting device to shave or debride tissue within a patient's sinuses. In addition to or in lieu of having the components and operability described herein IGS navigation system (100) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein.

IGS navigation system (100) of the present example comprises a field generator assembly (200), which comprises set of magnetic field generators (206) that are integrated into a horseshoe-shaped frame (204). Field generators (206) are operable to generate alternating magnetic fields of different frequencies around the head (H) of the patient (P). Navigation guidewire (130) may be a standalone device or may be positioned on an end effector or other location of a medical instrument such as a surgical cutting instrument or dilation instrument. In the present example, frame (204) is mounted to a chair (300), with the patient (P) being seated in the chair (300) such that frame (204) is located adjacent to the head (H) of the patient (P). By way of example only, chair (300) and/or field generator assembly (200) may be configured and operable in accordance with at least some of the teachings of U.S. patent application Ser. No. 15/933,737, entitled “Apparatus to Secure Field Generating Device to Chair,” filed Mar. 23, 2018, the disclosure of which is incorporated by reference herein.

IGS navigation system (100) of the present example further comprises a processor (110), which controls field generators (206) and other elements of IGS navigation system (100). For instance, processor (110) is operable to drive field generators (206) to generate alternating electromagnetic fields; and process signals from navigation guidewire (130) to determine the location of a sensor in navigation guidewire (130) within the head (H) of the patient (P). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

Navigation guidewire (130) includes a sensor (not shown) that is responsive to positioning within the alternating magnetic fields generated by field generators (206). A coupling unit (132) is secured to the proximal end of navigation guidewire (130) and is configured to provide communication of data and other signals between console (116) and navigation guidewire (130). In the present example, the sensor of navigation guidewire (130) comprises at least one coil at the distal end of navigation guidewire (130). When such a coil is positioned within an alternating electromagnetic field generated by field generators (206), the alternating magnetic field may generate electrical current in the coil, and this electrical current may be communicated along the electrical conduit(s) in navigation guidewire (130) and further to processor (110) via coupling unit (132). This phenomenon may enable IGS navigation system (100) to determine the location of the distal end of navigation guidewire (130) or other medical instrument (e.g., dilation instrument, surgical cutting instrument, etc.) within a three-dimensional space (i.e., within the head (H) of the patient (P), etc.). To accomplish this, processor (110) executes an algorithm to calculate location coordinates of the distal end of navigation guidewire (130) from the position related signals of the coil(s) in navigation guidewire (130).

Processor (110) uses software stored in a memory of processor (110) to calibrate and operate IGS navigation system (100). Such operation includes driving field generators (206), processing data from navigation guidewire (130), processing data from operating controls (112), and driving display screen (114). In some implementations, operation may also include monitoring and enforcement of one or more safety features or functions of IGS navigation system (100). Processor (110) is further operable to provide video in real time via display screen (114), showing the position of the distal end of navigation guidewire (130) in relation to a video camera image of the patient's head (H), a CT scan image of the patient's head (H), and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head (H), such as navigation guidewire (130), such that the operator may view the virtual rendering of the instrument at its actual location in real time. By way of example only, display screen (114) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (114).

The images provided through display screen (114) may help guide the operator in maneuvering and otherwise manipulating instruments within the patient's head (H). It should also be understood that other components of surgical instrument (10), described below, may incorporate a sensor like the sensor of navigation guidewire (130), including but not limited to shaft assembly (16).

II. Exemplary Surgical Cutting Instrument

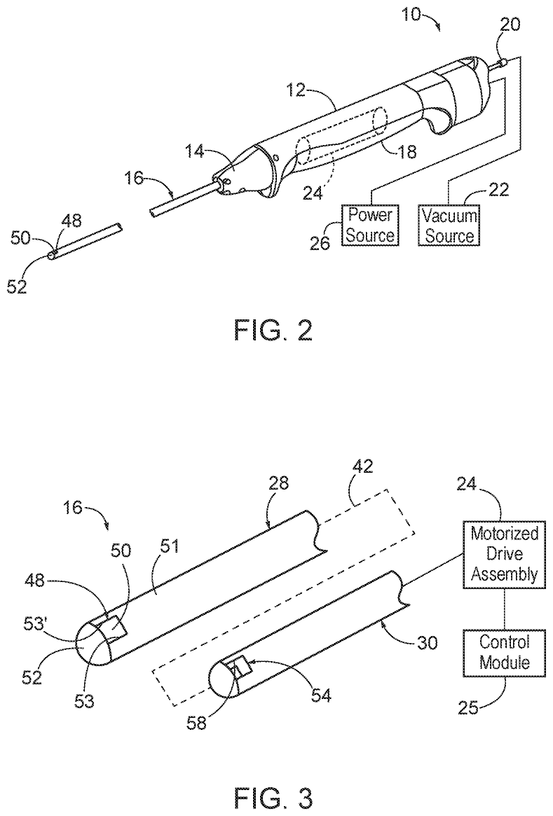

FIGS. 2-3 show a surgical cutting instrument (10) that may be used to remove tissue, such as bone tissue, from the nasal cavity, as well as from any other suitable location. Surgical cutting instrument (10) may be used in conjunction with an IGS navigation system such as IGS navigation system (100). Surgical cutting instrument (10) of the present example includes a handle assembly (12), a hub (14), and a first shaft assembly (16) extending distally from handle assembly (12). Handle assembly (12) has a handle (18) which may be of any suitable configuration. Handle (18) may include controls for the operation of surgical cutting instrument (10), or the controls may be located remotely. Surgical cutting instrument (10) further includes a suction port (20) operatively connected to a vacuum source (22) and configured to enable aspiration of tissue, such as a bone tissue, from a surgical site. Rotational motion is delivered by a motorized drive assembly (24) within handle assembly (12) to shaft assembly (16) in the present example. A power source (26) connects to motorized drive assembly (24) to power surgical cutting instrument (10) for use.

Shaft assembly (16) generally includes an outer shaft (28) and an inner cutting member (30) collectively configured to receive and remove tissue from the surgical site. Cutting member (30), which is illustrated as a tube, is disposed within a longitudinally extending lumen (32) of shaft (28). Cutting member (30) is configured to be rotated about a longitudinal axis (42) of shaft assembly (16) at a distal portion. Although shaft assembly (16) is depicted as rigid, all or a portion of shaft assembly (16) may be flexible. Cutting member (30) defines a lumen and extends proximally to handle assembly (12) and connects to motorized drive assembly (24), which rotatably drives cutting member (30) relative to shaft (28).

Shaft (28) includes a window region (48) having a shaft window opening (50). A tubular sidewall (51) distally terminates in a generally hemispherical end (52). Shaft window opening (50) extends through tubular sidewall (51) of shaft (28) into central lumen (40) and is in fluid communication with the environment surrounding shaft (28). Shaft window opening (50) faces radially outwardly relative to longitudinal axis (42) such that tissue is configured to be radially received through shaft window opening (50) into central lumen (40) in a radially inward direction. Shaft window opening (50) is surrounded by a relatively dull edge (53).

Cutting member (30) includes a cutting window opening (54) at distal portion of cutting member (30). Cutting window opening (54) is configured to longitudinally align with shaft window opening (50) and includes a cutting edge (58) extending therealong. At least a portion of cutting edge (58) is disposed to move adjacent to and across at least a portion of window region (48) when cutting member (30) is rotated or oscillated about longitudinal axis (42). By way of example, as cutting member (30) moves in a clockwise direction, edge (53) of window region (48) provides an opposing surface to cutting edge (58) whereby tissue may be severed to remove a cut tissue portion therefrom.

With continued reference to FIGS. 2-3, vacuum source (22) generates suction in a proximal direction along longitudinal axis (42) toward suction port (20). Once tissue is respectively introduced into window opening (54), suction effectively draws tissue into window opening (54) for resection while tissue blocks airflow along lumen. Additional details regarding airflow through lumen and aspiration vents for improving such airflow are discussed in alternative examples described in U.S. patent application Ser. No. 15/795,473, entitled “Tissue Shaving Instrument,” filed Oct. 27, 2017, the disclosure of which is incorporated by reference herein.

Control module (25) may be contained with handle assembly (12) and is electrically connected to motorized drive assembly (24), which drives rotation of inner cutting member (30). Based on signals from controls of the surgical cutting instrument (10), signals from IGS navigation system (100), or signals from another device, sensor, or source, control module (25) thereby directs rotation of inner cutting member (30) to either cease driving rotation of inner cutting member (30) or simply prevent rotation regardless of input provided by the operator.

III. Exemplary System and Method for Fail-Safe Tissue Removal

During FESS procedures and other surgical procedures, medical instruments such as surgical cutting instrument (10) may be used to remove bone and tissue. Due to the high speed at which tissue is removed using surgical cutting instrument (10), as well as the limited visibility and cramped quarters of the procedure area, it may be desirable to perform such removal with a high level of accuracy and safety. Otherwise, there may be a risk of inadvertent damage to delicate anatomical structures in or near the nasal cavity of the patient. Accordingly, it may be advantageous to provide a system and method to minimize and potentially eliminate the patient risks associated with tissue removal.

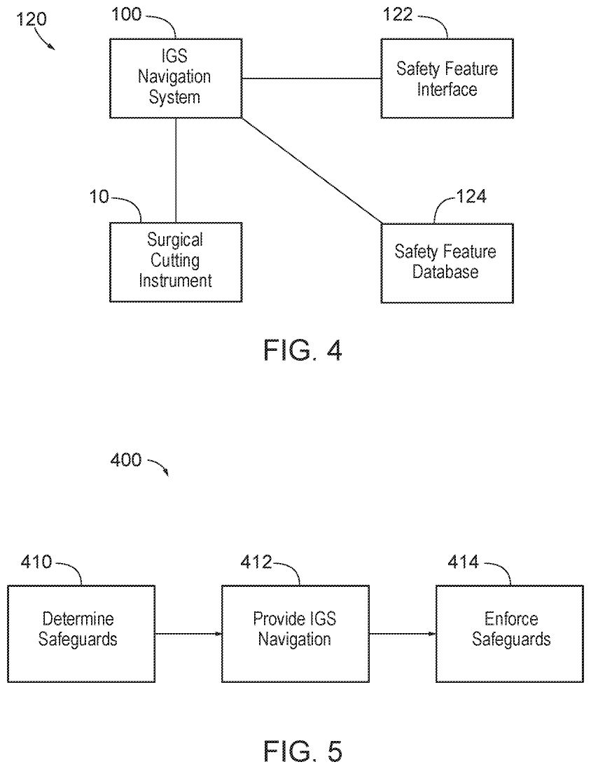

FIG. 4 shows an exemplary system (120) configured to provide one or more safety features during a surgical procedure. The system (120) shown comprises the surgical cutting instrument (10), which is usable with the IGS navigation system (100) during procedures as described above. In addition to being tracked by the IGS navigation system (100), the surgical cutting instrument (10) is in communication with the IGS navigation system (100) via one or more components such as control module (25) or power source (26), such that signals provided by the IGS navigation system (100) can control the function of the surgical cutting instrument (10).

The system (120) also comprises a safety feature interface (122), which may include a user interface for interacting with and configuring safety features of the system (120), and that is provided by a software application or website accessed by a computer, tablet, smartphone, or other computing device. In some versions, safety feature interface (122) is provided via display screen (114) and operating controls (112). The system (120) also comprises a safety feature database (124) that is configured to store data related to safe operation of the surgical cutting instrument (10). The safety feature database (124) may be accessible by the IGS navigation system (100) over a wired or wireless network; or may be locally stored on the IGS navigation system (100) or another device in communication with the system (120); or both. In the shown implementation, the system (120) is configured to allow the IGS navigation system (100) to control the operation and performance of the surgical cutting instrument (10) based upon a tracked position, orientation, and movement of the surgical cutting instrument (10) and one or more safety features that are configured by a user of the feature interface (122), stored in the safety feature database (124), or both. Operating in this manner, when the IGS navigation system (100) determines that the surgical cutting instrument (10) is being used in a manner that presents an unnecessary risk to a patient, the IGS navigation system (100) may provide alerts or vary the operation of the surgical cutting instrument (10) in order to call attention to or reduce the risks.

As will be apparent to one skilled in the art in light of this disclosure, the components of the system (120) could be arranged differently while still preserving the function of the system (120). For example, in some implementations, the safety feature interface (122) and the safety feature database (124) may be directly in communication with the surgical cutting instrument (10), and the control module (25) may receive position and movement information from the IGS navigation system (100), and safety data from the safety feature interface (122) and the safety feature database (124), and may vary its own operation based thereupon.

FIG. 5 shows an exemplary set of high level steps (400) that may be performed by the system (120) to provide one or more safety features during a procedure using the IGS navigation system (100) and the medical instrument (10). These high-level steps include determining (block 410) one or more safeguards based upon user configurations or default settings, providing (block 412) IGS navigation and tracking the medical instrument (10) throughout, and enforcing (block 414) the one or more safeguards throughout the procedure based upon tracking the medical instrument (10). Operating in this manner, the system (120) may reduce patient risks during a procedure without requiring additional configuration by a physician and, unless and until safeguards are triggered and enforced (block 414), operating in a substantially undetectable manner since no outwardly apparent modification to the medical instrument (10), IGS navigation system (100), or other system components are required. Exemplary implementations of the set of high level steps (400) will be discussed in more detail below.

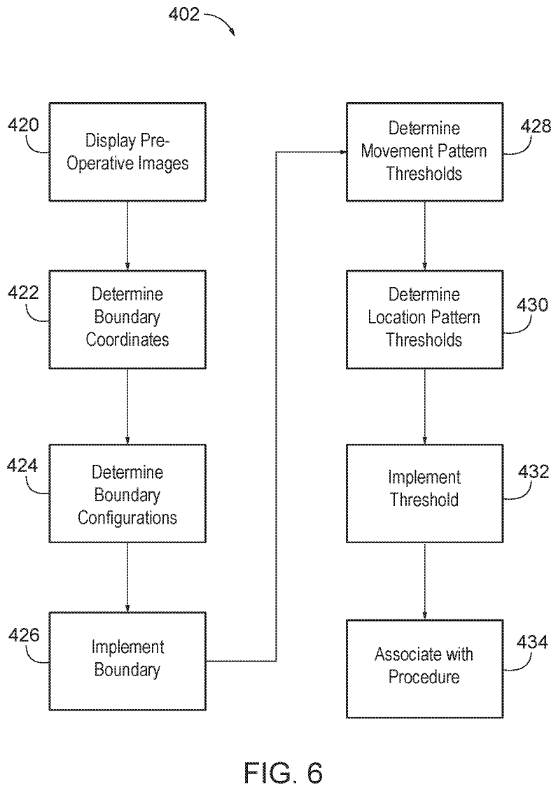

For example, FIG. 6 depicts an exemplary set of steps (402) that may be performed by the system (120) to configure the one or more safety features. The exemplary steps (402) include displaying (block 420) a set of pre-operative images via the safety feature interface (122) to allow a physician or other user to review the images and, if desired, provide boundaries and boundary configurations via the safety feature interface (122) by marking one or more areas of the images to indicate that they are areas where the medical instrument (10) should not be used, or used only with additional caution. The system (120) may then use these inputs to determine (block 422) boundary coordinates, determine (block 424) boundary configurations, and implement (block 426) one or more boundaries. A boundary implemented (block 426) in this manner may be, for example, a positive boundary (e.g., an area of the pre-operative images that the medical instrument (10) should stay within) or a negative boundary (e.g., an area of the pre-operative images that the medical instrument (10) should not enter; or should only enter with caution). Boundaries may take a variety of forms, and could include, for example, a two-dimensional plane or a three-dimensional object or area located within the three-dimensional space of the operative area.

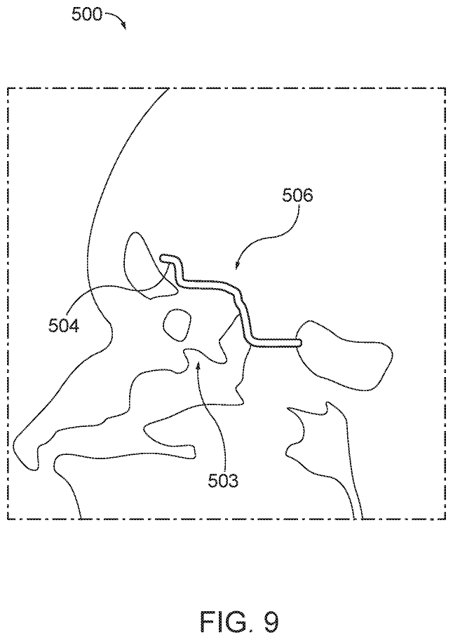

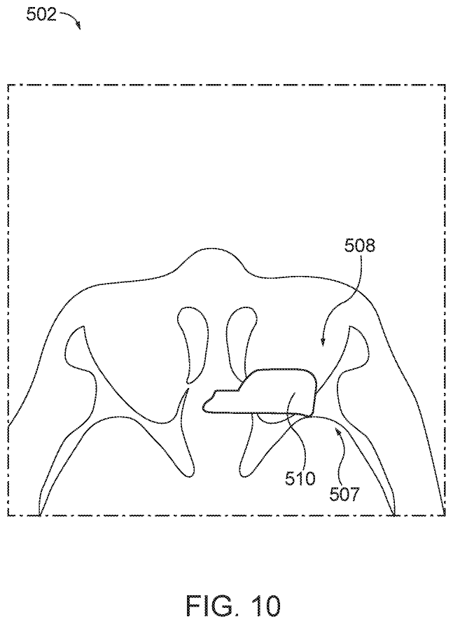

FIGS. 9-10 each show screenshots of an exemplary interface that may be used to configure a set of one or more boundaries to be implemented (block 426) by the system (120). These interfaces may be rendered through safety feature interface (122) or otherwise. FIG. 9 shows a pre-operative image (500) of a side cross-sectional view of a patient's sinus area, which may be viewed prior to a procedure performed in that area. A boundary line (504) has been manually added by a user via the safety feature interface (122), defining a boundary between a low risk area (503) and a high-risk area (506). Similarly, FIG. 10 shows a pre-operative image (502) of a top-down cross-sectional view of a patient's sinus area, with a boundary area (510) located between a low-risk area (508) and a high-risk area (507); or, in some implementations, defining an area within the boundary area (510) itself that is a high-risk area. The boundary line (504) or boundary area (510) may be entered by the user in a variety of ways by interacting with the displayed image, for example by using a keyboard and mouse (e.g., operating controls (112)), an interactive touchscreen (e.g., display screen (114)), a digital stylus, or a virtual reality wand controller of the safety feature interface (122).

After a boundary line (504) or boundary area (510) is initially defined, a user may provide additional information via the safety feature interface (122) to allow the system (120) to finish determining (block) 422 the boundary coordinates. This could include providing and associating a depth measurement with a boundary line (504); or providing and associating a depth measurement with a boundary area (510). The boundary line (504) of FIG. 9 defines a boundary for the single pre-operative image (500); but by providing a depth for that boundary line (504), the line may be extended into other pre-operative images that fall above or below the single pre-operative image where the line is initially provided. For example, if there is a set of one hundred pre-operative images showing the side cross-sectional view of the sinus area at various depths, providing a depth measurement could allow the boundary line (504) to extend through all one hundred images (e.g., forming an unbroken boundary area that covers the entire width of the patient's sinus area past a certain depth when viewed from the front of the patient sinus area rather than the side), or extending through less than a hundred of the images (e.g., forming an isolated boundary area that covers a portion of the width of the patient's sinus area past a certain depth when viewed from the front of the patient sinus area rather than the side).

The result is similar when providing and associating a depth with a boundary area (510). With reference to FIG. 10, it can be seen that the boundary area (510) defines a high-risk area (e.g., either high-risk area (507), or the area within the boundary area (510), or both), at the depth of that pre-operative image (502). By providing a depth, the boundary area (510) can be extended upward, downward, or both through a set of pre-operative images having the same top-down view of the sinus area as the pre-operative image (502). In this manner, a three-dimensional boundary can be created from the boundary area (510), which may be useful when there is a region of an operative area that is high risk but is surrounded by low risk areas that may be involved in a procedure. As with previous examples, a boundary area (510) can be extended through an entire set of images, or through less than the entire set of images, as may be desirable for a particular circumstance.

As an alternative to providing an explicit depth or width to associate with a boundary line (504) or boundary area (510), the system (120) may also allow a user to link boundaries added to two or more pre-operative images together to create a boundary. For example, a user may draw a first boundary line (504) on a pre-operative image (500) at a first depth, then navigate to a pre-operative image of the same set at a different depth, then draw a second boundary line. The system (120) may then connect the first boundary line (504) and the second boundary line such that it forms a boundary area stretching from the first boundary line (504) to the second boundary line through the set of pre-operative images. By adding boundary lines to a plurality of images within a stack of pre-operative images (with the pre-operative images representing adjacent cross-sectional planes of the patient's anatomy), various levels of boundary curvature could be achieved. The above principal could also be applied to link a boundary line (504) to a boundary area (510) to create a planar boundary area that varies through the depth of a set of pre-operative images (e.g., having a top-down profile of the boundary area (510), and a side profile of the boundary line (504)). Similarly, a boundary area (510) could be linked with one or more other boundary areas at various depths of a stack of pre-operative images to create three-dimensional boundaries of various shapes. Other similar ways in which boundary lines, boundary areas, width measurements, depth measurements, and other user inputs may be used individually or in combination with each other to produce various boundaries within a set of pre-operative images exist and will be apparent to one skilled in the art in light of this disclosure.

It should also be understood that, in addition to determining (block 422) boundary coordinates based upon manual inputs as described above, the system (120) may also determine (block 422) boundary coordinates for a procedure by automatically generating coordinates based upon object recognition analysis of pre-operative images to uniquely identify to high risk areas, or by selecting boundary coordinates from a source such as the safety feature database (124) and automatically applying them to a set of pre-operative images, or both. In other words, the system (120) may be able to recognize high risk anatomical structures within the sets of pre-operative images; and may identify such anatomical structures as high risk structures in rendering feedback to the end user.

With one or more boundaries determined (block 422), the system (120) may also determine (block 424) boundary configurations for the boundaries based upon user inputs via the safety feature interface (122), configurations stored in the safety featured database (124), or both. Regardless of the source, this could include, for example, configuring the type of boundary (e.g., a positive boundary that the medical instrument (10) can safely operate within or a negative boundary that the medical instrument (10) should not breach), and the result of nearing or breaching the boundary with the medical instrument (10) (e.g., providing a first visual or audible notification upon nearing, providing a second visual or audible notification upon breaching, or reducing or terminating operation of the medical instrument (10) entirely upon nearing or breaching).

With one or more boundaries determined (block 422) and configured (block 424), the system (120) may then implement (block 426) the boundaries for one or more associated procedures. Boundary implementation (block 426) may vary by a particular implementation of the disclosed technology but should be understood to include performance of necessary steps to ready the configured boundaries for use and enforcement during a procedure. This could include, for example, propagating the boundary across a number of pre-operative images, compiling the boundary or converting the boundary into a different format, and making the boundary available for use during the procedure by storing it on one or more of the safety feature database (124), the IGS navigation system (100), or the surgical cutting instrument (10).

As part of determining (block 410) the safeguards, the system (120) may also determine (block 428) one or more movement pattern thresholds and may determine (block 430) one or more location pattern thresholds, either based upon manual input from a user via the safety feature interface (122), or by retrieval from the safety feature database (124), or both. Movement pattern thresholds could include unsafe patterns that may be identified based upon movement information from the surgical cutting instrument (10) (e.g., from an accelerometer or gyroscope, etc.) or IGS navigation system (e.g., from navigation guidewire (130) tracking, etc.), or both.

Unsafe movement patterns could include, for example, those identified as exceeding a maximum linear movement speed or acceleration, exceeding a maximum rotational (e.g., roll, pitch, or yaw) speed, acceleration, or displacement (especially with respect to pitch or yaw) of the handle assembly (12) of the surgical cutting instrument, and exceeding a maximum vibration strength or frequency, any of which may indicate an unintentional, reactive, or otherwise undesirable movement of the surgical cutting instrument (10) during a procedure. Such movements may result from human error or other factors (e.g., dropping the surgical cutting instrument (10) or a sudden sneeze or muscle spasm), environmental factors (e.g., having an arm jostled by a nearby person or a device or other equipment suddenly shifting), mechanical errors or other factors (e.g., a malfunction of some component of the surgical cutting instrument (10)), or other sources. Such pattern detection thresholds allow the system (120) to detect such conditions and reduce patient risks associated with them by providing notifications or modifying performance of surgical cutting instrument (10). In addition to determining such patterns, configurations for actions resulting from detection may also be configured or retrieved, including the types and characteristics of notifications provided, and the types and characteristics of device control performed.

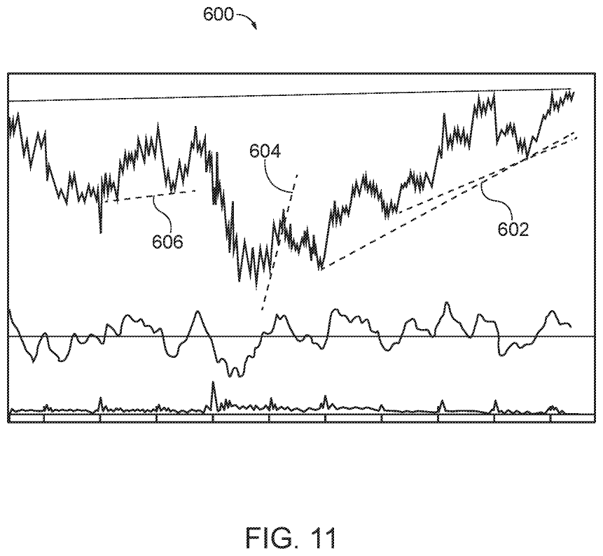

One method that may be advantageous for efficiently detecting unsafe movement patterns would be to store and analyze procedure data related to movements of the surgical cutting instrument (10) as a real-time data graph; and identify unsafe activity by examining the slope of the graph within various periods of time. FIG. 11 shows a visualization of such a graph (600) that may be appropriate for linear movements over time. That graph (600) show a first time period (606) with linear speed having a relatively moderate upward slope over a moderate period of time that the system (120) will determine is a safe movement speed. A second time period (604) shows a steeper linear speed increase, but due to the brief period of time over which it occurs, the system (120) will determine that it is a safe movement speed. However, during a third time period (602), the system will identify a relatively steep upward slope for linear speed over a relatively long period of time and will determine that this is an unsafe movement. Other methods for efficiently analyzing movement patterns exist and will be apparent to one skilled in the art in light of this disclosure.

Unsafe location patterns could include, for example, patterns that result in the surgical cutting instrument (10) being present within or steadily moving towards a high-risk location (e.g., a location manually configured as a boundary, or locations automatically configured as high risk due to proximity to the carotid artery or other artery), patterns that result in the surgical cutting instrument (10) steadily deviating from a predicted area or path associated with the procedure, or patterns that result in the surgical cutting instrument (10) remaining within a certain area for an undue amount of time, any of which may indicate an unintentional or otherwise undesirable use of the surgical cutting instrument (10) during a procedure. As with above, such location patterns may result from human error or other factors, environment factors, mechanical error or other factors, or other sources. Detection of such undesirable location patterns allow the system (120) to detect conditions that may not be identified based upon individual unsafe movements or other characteristics and reduce patient risks associated with them by providing notifications or modifying performance of surgical cutting instrument (10). In addition to determining such patterns, configurations for actions resulting from detection may also be configured or retrieved, including the types and characteristics of notifications provided, and the types and characteristics of surgical cutting instrument (10) control performed.

With one or more thresholds determined (block 428, block 430), the system (120) may implement (block 432) those thresholds similarly to the implementation (block 426) of boundaries. This could include compiling or converting the thresholds into a different format and making the boundary available for use during the procedure by storing it on one or more of the safety feature database (124), the IGS navigation system (100), or the surgical cutting instrument (10). With boundaries and thresholds prepared for use, the system (120) may then associate (block 434) the prepared safety features with one or more procedures. This could include configuring the system (120) to automatically prepare and enable the safety features when the IGS navigation system (100) is activated for an associated procedure; or configuring the system (120) to make the safety features available or selectable when the IGS navigation system (100) is activated for an associated procedure, or both.

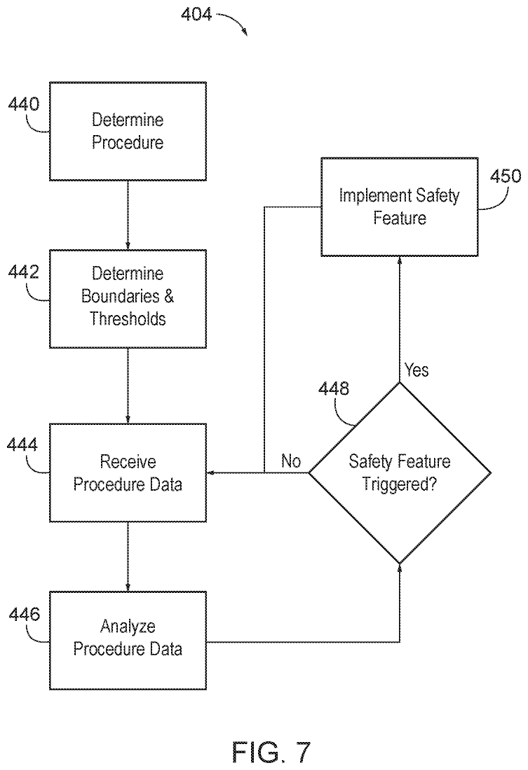

FIG. 7 shows an exemplary set of steps (404) that may be performed by the system (120) to monitor for conditions that are covered by the one or more safety features. When the IGS navigation system (100) is activated or enable for a particular procedure, the system (120) may determine (block 440) a unique identifier for that procedure; and determine (block 442) any boundaries or thresholds associated with that procedure. As has been discussed, procedure information and prepared boundaries and thresholds may be available and accessible on one or more of the safety feature database (124), the IGS navigation system (100), or the surgical cutting instrument (10) itself as may be desirable for a particular implementation. As one example, this may then include the IGS navigation system (100) itself determining the procedure (block 440), and then identifying and determining (block 442) locally stored boundaries and thresholds for the procedure.

During the IGS navigation system (100) enhanced procedure, the system (120) will receive (block 444) procedure data from various sources (e.g., from the surgical cutting instrument (10) or from the IGS navigation system (100)) and will analyze (block 446) that procedure data. Analyzing (block 446) procedure data may include individually examining each discrete piece of information generated during the procedure and comparing it against all safety features, categorizing data as it arrives and only comparing it against safety features associated with that category of data, prioritizing data s it arrives and comparing higher priority data against safety features as processing time becomes available, or other similar methods. If no safety features are triggered (block 448) or otherwise implicated by analysis (block 446) of procedure data, the system (120) will take no action and continue to receive (block 444) and analyze (block 446) data throughout the procedure. If a safety feature is triggered (block 448) or otherwise implicated by analyzed (block 446) data, the system (120) will implement (block 450) the safety feature by performing one or more actions configured for the triggered (block 448) safety feature, and then continue to receive (block 444) additional procedure data. As one example, this could include the IGS navigation system (100) detecting the breach or proximity of the surgical cutting instrument (10) to a boundary based upon its own locally available location information; and reducing the cutting speed or disabling the cutting action entirely by providing a signal to the power source (26) or control module (25) in response.

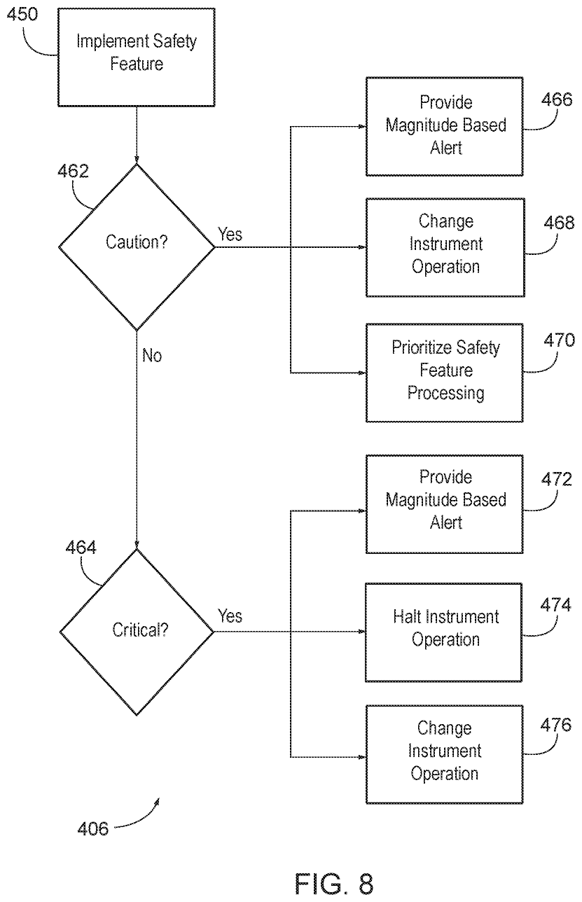

As another example, FIG. 8 depicts an exemplary set of steps (406) that may be performed by the system (120) to address various conditions that are covered by the one or more safety features. After the system (120) has determined that a safety feature should be implemented (block 450), the system (120) may determine whether the safety feature is a cautionary feature (block 462) or a critical feature (block 464) based upon the safety features configurations or a real-time assessment, and then take appropriate action based upon the safety features configurations or a real-time assessment. For example, in some situations a safety feature associated with a particular boundary may be configured as a cautionary feature, but in certain circumstances (e.g., where the surgical cutting instrument (10) is currently activated at a high cutting speed) the system (120) may interpret the safety feature as a critical feature. Continuing the same example, the resulting action for the safety feature may be configured to provide an audible alert, but in the same circumstances (e.g., when the surgical cutting instrument (10) is activated at a high cutting speed) the system (120) may determine in real time that the cutting speed should be reduced.

Reactions to cautionary and critical safety features may vary widely, but cautionary alert reactions may include, for example, providing (block 466) a magnitude-based alert such as increasingly bright visual alerts or increasingly loud audible alerts, changing (block 468) the operation of surgical cutting instrument (10) such as reducing cutting speed or power, or prioritizing (block 470) future processing of procedure data related to the cautionary safety feature until the risk subsides. This could include identifying the particular safety feature and procedure data that led to the cautionary (block 462) determination, and flagging all incoming data related to that safety feature and type of data for prioritized processing, whether by the same processor or a dedicated high priority processer, in order to more immediately detect if the cautionary (block 462) feature elevates to a critical (block 464) feature. As an example, if the surgical cutting instrument (10) is proceeding towards a boundary but is still a safe distance away, the system (120) may provide cautionary (block 462) features such providing (block 466) magnitude based audible alerts and prioritizing (block 470) future processing. As the surgical cutting instrument (10) breaches the boundary, the procedure data indicating as such may be prioritized and processed near immediately in order to determine that it is a critical (block 464) feature so that the instrument operation may be halted (block 474) entirely.

Other reactions to critical (block 464) features beyond halting (block 474) surgical cutting instrument (10) operation entirely may include, for example, providing (block 472) additional magnitude-based alerts of increasing magnitude or differing types (e.g., haptic feedback) or changing (block 476) operation of the surgical cutting instrument (10) further (e.g., further reducing cutting power or otherwise reducing features available via the surgical cutting instrument (10)). Reactions available to a particular implementation may vary based upon that implementation and the particular devices and surgical instruments being used, these and other appropriate reactions to both cautionary and critical features will be apparent to one skilled in the art in light of this disclosure.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: a surgical instrument; and an image guided surgery navigation system configured to track the position of at least one portion of the surgical instrument in order to produce a set of procedure data during a procedure; wherein the image guided surgery navigation system is further configured to: receive a set of safety feature definitions associated with locations within anatomy of a patient, as the set of procedure data is produced, compare the set of procedure data to the set of safety feature definitions to determine if any portion of the set of procedure data indicates any risk factors associated with the surgical instrument based on the tracked position of the at least one portion of the surgical instrument within the anatomy of the patient, and where a risk factor is indicated, perform a risk mitigation task based upon that risk factor.

### EXAMPLE 2

The apparatus of Example 1, the apparatus further comprising a safety feature interface that is configurable on a user device, wherein: the set of safety feature definitions comprises a set of boundaries received via the safety feature interface, wherein each boundary of the set of boundaries defines a high-risk area of at least one preoperative image of a set of pre-operative images of the anatomy of the patient, and wherein the image guided surgery navigation system is further configured to indicate the risk factor when the set of procedure data indicates that the surgical instrument is proximate to the high-risk area of a boundary.

### EXAMPLE 3

The apparatus of Example 2, wherein the safety feature interface is configured to receive input defining a boundary line of the set of boundaries.

### EXAMPLE 4

The apparatus of Example 3, wherein the safety feature interface is configured to receive the input defining the boundary line as a drawn line via a touchscreen input of a user device on which the safety feature interface is configured.

### EXAMPLE 5

The apparatus of any one or more of Examples 2 through 4, wherein the safety feature interface is configured to: receive input defining a boundary line of the set of boundaries and a depth associated with the boundary line, and apply the boundary line to the set of pre-operative images based on the depth to produce a boundary area and add the boundary area to the set of boundaries.

### EXAMPLE 6

The apparatus of any one or more of Examples 2 through 5, wherein the safety feature interface is configured to: receive input defining a boundary area of the set of boundaries and a depth associated with the boundary area, and apply the boundary area to the set of pre-operative images based on the depth to produce a three-dimensional boundary area and add the three-dimensional boundary area to the set of boundaries.

### EXAMPLE 7

The apparatus of any one or more of Examples 2 through 6, wherein the safety feature interface is configured to: receive input defining a first boundary on a first pre-operative image of the set of preoperative images and a second boundary on a second pre-operative image of the set of preoperative images, and produce a third boundary by connecting the first boundary with the second boundary across at least one intervening image of the set of pre-operative images.

### EXAMPLE 8

The apparatus of any one or more of Examples 1 through 7, wherein the set of safety feature definitions comprise a set of movement pattern thresholds, wherein each of the set movement pattern thresholds defines a high-risk movement associated with the surgical instrument, and wherein the image guided surgery navigation system is further configured to indicate the risk factor when the set of procedure data indicates that the surgical instrument has performed the high-risk movement.

### EXAMPLE 9

The apparatus of Example 8, wherein the set of movements patterns is configured to detect at least one of: a linear speed of the surgical instrument exceeding a speed threshold, a vibration frequency of the surgical instrument exceeding a vibration threshold, or a rotational displacement of the surgical instrument exceeding a rotational threshold.

### EXAMPLE 10

The apparatus of any one or more of Examples 8 through 9, wherein the image guided surgery navigation system is further configured to use at least one movement pattern threshold of the set of movement pattern thresholds to: graph a linear speed of the surgical instrument over time based on the set of procedure data, determine a slope threshold for a period of time of the graph, and indicate the risk factor when a slope associated with linear speed exceeds the slope threshold for the period of time.

### EXAMPLE 11

The apparatus of any one or more of Examples 1 through 10, wherein the set of safety feature definitions comprise: a set of boundaries indicating one or more high risk areas on a set of pre-operative images, a set of movement patterns indicating high risk movements of the surgical device, and a set of risk mitigation tasks, wherein each of the set of boundaries and each of the set of movements patterns is associated with at least one risk mitigation task.

### EXAMPLE 12

The apparatus of any one or more of Examples 1 through 11, wherein the set of risk mitigation tasks comprises one or more of: an audible alert configured to produce a sound when performed, a visual alert configured to produce a light when performed, or an instrument operation override configured to change the operation of the surgical instrument when performed.

### EXAMPLE 13

The apparatus of any one or more of Examples 1 through 12, wherein the set of risk mitigation tasks comprise a prioritized processing task, and wherein the prioritized processing task is configured to, when performed, cause the image guided surgery navigation system to: determine a subset of procedure data from the set of procedure data and a safety feature definition of the set of safety feature definitions that caused the prioritized processing task to be performed, and prioritize the processing of a set of subsequent procedure data that is associated with the subset of procedure data and the safety feature definition.

### EXAMPLE 14

The apparatus of any one or more of Examples 1 through 13, wherein the surgical instrument comprises a surgical cutting instrument.

### EXAMPLE 15

A method for reducing patient risk associated with the use of a surgical instrument during a procedure comprising the steps: displaying a set of pre-operative images to a physician, the pre-operative images depicting anatomical structures of a patient; receiving a set of boundaries via a safety feature interface, wherein each boundary of the set of boundaries is associated with one or more anatomical structures depicted in one or more of the set of pre-operative images; tracking a position of at least one portion of the surgical instrument with an image guided surgical navigation system during the procedure and producing a set of procedure data based thereon; comparing the set of procedure data to the set of boundaries to determine whether the surgical instrument is proximate to any boundary in the set of boundaries; and where any portion of the set of procedure data indicates that the surgical instrument is proximate to a boundary, perform a risk mitigation task associated with that boundary.

### EXAMPLE 16

The method of Example 15, further comprising the steps of, when receiving the set of boundaries: receiving the set of boundaries as a set of lines drawn on one or more of the set of pre-operative images via a touchscreen input of the safety feature interface; and for each boundary in the set of boundaries, determining whether that boundary is a line boundary or an area boundary.

### EXAMPLE 17

The method of any one or more of Examples 15 through 16, further comprising the steps of: receiving a set of risk mitigation tasks; and associating each of the set of boundaries with at least one risk mitigation task in the set of risk mitigation tasks, wherein the set of risk mitigation tasks comprises one or more of: an audible alert configured to produce a sound when performed, or an instrument operation override configured to change the operation of the surgical instrument when performed.

### EXAMPLE 18

An image guided surgery navigation system configured to track the location of a surgical instrument during a procedure in order to produce a set of procedure data, wherein the image guided surgery navigation system is further configured to: receive a set of safety feature definitions, as the set of procedure data is produced, compare the set of procedure data to the set of safety feature definitions to determine if any portion of the set of procedure data indicates any risk factors associated with a position of the surgical instrument within a patient, and where a risk factor is indicated based on the position of the surgical instrument within the patient, perform a risk mitigation task based upon that risk factor.

### EXAMPLE 19

The image guided surgery navigation system of Example 18, the set of safety feature definitions comprising a set of boundaries, wherein each boundary in the set of boundaries defines a high-risk area of an anatomical structure depicted in at least one pre-operative image of a set of pre-operative images associated with the procedure, wherein the image guided surgery navigation system is further configured to: receive the set of boundaries as a set of lines drawn on one or more of the set of pre-operative images via a touchscreen input of a safety feature interface, and indicate the risk factor when the set of procedure data indicates that the surgical instrument is proximate to the high-risk area of a boundary.

### EXAMPLE 20

The image guided surgery navigation system of Example 19, the set of safety feature definitions further comprising a set of movement patterns, wherein each movement pattern in the set of movement patterns defines a high-risk movement associated with the surgical instrument, wherein the image guided surgery navigation system is further configured to indicate the risk factor when the set of procedure data indicates that the surgical instrument has performed the high-risk movement, wherein the set of movement patterns is configured to detect at least a linear speed of the surgical instrument exceeding a speed threshold.

V. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a surgical instrument; and
(b) an image guided surgery navigation system configured to track the position of at least one portion of the surgical instrument in order to produce a set of procedure data during a procedure, wherein the image guided surgery navigation system is further configured to:
(i) receive a set of safety feature definitions associated with locations within anatomy of a patient,
(ii) as the set of procedure data is produced, compare the set of procedure data to the set of safety feature definitions to determine if any portion of the set of procedure data indicates any risk factors associated with the surgical instrument based on the tracked position of the at least one portion of the surgical instrument within the anatomy of the patient, and
(iii) where a risk factor is indicated, perform a risk mitigation task based upon that risk factor.

2. The apparatus of claim 1, the apparatus further comprising a safety feature interface that is configurable on a user device, wherein the set of safety feature definitions comprises a set of boundaries received via the safety feature interface, wherein each boundary of the set of boundaries defines a high-risk area of at least one preoperative image of a set of pre-operative images of the anatomy of the patient, and wherein the image guided surgery navigation system is further configured to indicate the risk factor when the set of procedure data indicates that the surgical instrument is proximate to the high-risk area of a boundary.

3. The apparatus of claim 2, wherein the safety feature interface is configured to receive input defining a boundary line of the set of boundaries.

4. The apparatus of claim 3, wherein the safety feature interface is configured to receive the input defining the boundary line as a drawn line via a touchscreen input of a user device on which the safety feature interface is configured.

5. The apparatus of claim 2, wherein the safety feature interface is configured to:
(i) receive input defining a boundary line of the set of boundaries and a depth associated with the boundary line, and
(ii) apply the boundary line to the set of pre-operative images based on the depth to produce a boundary area and add the boundary area to the set of boundaries.

6. The apparatus of claim 2, wherein the safety feature interface is configured to:
(i) receive input defining a boundary area of the set of boundaries and a depth associated with the boundary area, and
(ii) apply the boundary area to the set of pre-operative images based on the depth to produce a three-dimensional boundary area and add the three-dimensional boundary area to the set of boundaries.

7. The apparatus of claim 2, wherein the safety feature interface is configured to:
(i) receive input defining a first boundary on a first pre-operative image of the set of preoperative images and a second boundary on a second pre-operative image of the set of preoperative images, and
(ii) produce a third boundary by connecting the first boundary with the second boundary across at least one intervening image of the set of pre-operative images.

8. The apparatus of claim 1, wherein the set of safety feature definitions comprise a set of movement pattern thresholds, wherein each of the set movement pattern thresholds defines a high-risk movement associated with the surgical instrument, and wherein the image guided surgery navigation system is further configured to indicate the risk factor when the set of procedure data indicates that the surgical instrument has performed the high-risk movement.

9. The apparatus of claim 8, wherein the set of movements patterns is configured to detect at least one of:
(i) a linear speed of the surgical instrument exceeding a speed threshold,
(ii) a vibration frequency of the surgical instrument exceeding a vibration threshold, or
(iii) a rotational displacement of the surgical instrument exceeding a rotational threshold.

10. The apparatus of claim 8, wherein the image guided surgery navigation system is further configured to use at least one movement pattern threshold of the set of movement pattern thresholds to:
(i) graph a linear speed of the surgical instrument over time based on the set of procedure data,
(ii) determine a slope threshold for a period of time of the graph, and
(iii) indicate the risk factor when a slope associated with linear speed exceeds the slope threshold for the period of time.

11. The apparatus of claim 1, wherein the set of safety feature definitions comprise:
(i) a set of boundaries indicating one or more high risk areas on a set of pre-operative images,
(ii) a set of movement patterns indicating high risk movements of the surgical device, and
(iii) a set of risk mitigation tasks, wherein each of the set of boundaries and each of the set of movements patterns is associated with at least one risk mitigation task.

12. The apparatus of claim 1, wherein the set of risk mitigation tasks comprises one or more of:
(i) an audible alert configured to produce a sound when performed,
(ii) a visual alert configured to produce a light when performed, or
(iii) an instrument operation override configured to change the operation of the surgical instrument when performed.

13. The apparatus of claim 1, wherein the set of risk mitigation tasks comprise a prioritized processing task, and wherein the prioritized processing task is configured to, when performed, cause the image guided surgery navigation system to:
(i) determine a subset of procedure data from the set of procedure data and a safety feature definition of the set of safety feature definitions that caused the prioritized processing task to be performed, and
(ii) prioritize the processing of a set of subsequent procedure data that is associated with the subset of procedure data and the safety feature definition.

14. The apparatus of claim 1, wherein the surgical instrument comprises a surgical cutting instrument.

15. A method for reducing patient risk associated with the use of a surgical instrument during a procedure, the method comprising the steps:
(a) displaying a set of pre-operative images to a physician, the pre-operative images depicting anatomical structures of a patient;
(b) receiving a set of boundaries via a safety feature interface, wherein each boundary of the set of boundaries is associated with one or more anatomical structures depicted in one or more of the set of pre-operative images;
(c) tracking a position of at least one portion of the surgical instrument with an image guided surgical navigation system during the procedure and producing a set of procedure data based thereon;
(d) comparing the set of procedure data to the set of boundaries to determine whether the surgical instrument is proximate to any boundary in the set of boundaries; and
(e) where any portion of the set of procedure data indicates that the surgical instrument is proximate to a boundary, perform a risk mitigation task associated with that boundary.

16. The method of claim 15, further comprising the steps of, when receiving the set of boundaries:
(a) receiving the set of boundaries as a set of lines drawn on one or more of the set of pre-operative images via a touchscreen input of the safety feature interface; and
(b) for each boundary in the set of boundaries, determining whether that boundary is a line boundary or an area boundary.

17. The method of claim 15, further comprising the steps of:
(a) receiving a set of risk mitigation tasks; and
(b) associating each of the set of boundaries with at least one risk mitigation task in the set of risk mitigation tasks,
wherein the set of risk mitigation tasks comprises one or more of:
(i) an audible alert configured to produce a sound when performed, or
(ii) an instrument operation override configured to change the operation of the surgical instrument when performed.

18. An image guided surgery navigation system configured to track the location of a surgical instrument during a procedure in order to produce a set of procedure data, wherein the image guided surgery navigation system is further configured to:
(i) receive a set of safety feature definitions,
(ii) as the set of procedure data is produced, compare the set of procedure data to the set of safety feature definitions to determine if any portion of the set of procedure data indicates any risk factors associated with a position of the surgical instrument within a patient, and
(iii) where a risk factor is indicated based on the position of the surgical instrument within the patient, perform a risk mitigation task based upon that risk factor.

19. The image guided surgery navigation system of claim 18, the set of safety feature definitions comprising a set of boundaries, wherein each boundary in the set of boundaries defines a high-risk area of an anatomical structure depicted in at least one pre-operative image of a set of pre-operative images associated with the procedure, wherein the image guided surgery navigation system is further configured to:
(i) receive the set of boundaries as a set of lines drawn on one or more of the set of pre-operative images via a touchscreen input of a safety feature interface, and
(ii) indicate the risk factor when the set of procedure data indicates that the surgical instrument is proximate to the high-risk area of a boundary.

20. The image guided surgery navigation system of claim 19, the set of safety feature definitions further comprising a set of movement patterns, wherein each movement pattern in the set of movement patterns defines a high-risk movement associated with the surgical instrument, wherein the image guided surgery navigation system is further configured to indicate the risk factor when the set of procedure data indicates that the surgical instrument has performed the high-risk movement, wherein the set of movement patterns is configured to detect at least a linear speed of the surgical instrument exceeding a speed threshold.
